(19) United States
(12) Patent Application Publication
(10) Pub. No.: US 2022/0008742 A1
(43) Pub. Date: Jan. 13, 2022

(54) GLOBAL IRRADIATION THERMOTHERAPY SYSTEM BASED ON COORDINATED ROTATION OF MICROWAVE AND RADIO FREQUENCY, AND GLOBAL THERMOTHERAPY INSTRUMENT

(71) Applicant: Shengjie SHANG, Jieshou (CN)

(72) Inventors: Zhiying SHANG, Jieshou (CN); Chengde SHANG, Jieshou (CN); Shengjie SHANG, Jieshou (CN)

(21) Appl. No.: 17/485,610

(22) Filed: Sep. 27, 2021

Related U.S. Application Data
(63) Continuation of application No. PCT/CN2020/091361, filed on May 20, 2020.

(30) Foreign Application Priority Data
May 13, 2020 (CN) 202010403212.X

(51) Int. Cl. A61N 5/02; A61N 5/04; G01R 33/48

(52) U.S. Cl. CPC A61N 5/025; G01R 33/48; A61N 5/045

(57) ABSTRACT

A global irradiation thermotherapy system based on coordinated rotation of microwave and radio frequency, including an annular phantom, an intelligent control unit, a microwave rotary heating mechanism, a capacitive radio frequency rotary heating mechanism and a temperature-measuring mechanism connected with the intelligent control unit. This disclosure also provides a global thermotherapy instrument.

### CROSS-REFERENCE TO RELATED APPLICATIONS

This application is a continuation of International Patent Application No. PCT/CN2020/091361, filed on May 20, 2020, which claims the benefit of priority from Chinese Patent application No. 202010403212.X, filed on May 13, 2020. The content of the aforementioned applications, including any intervening amendments thereto, is incorporated herein by reference in its entirety.

### TECHNICAL FIELD

The present disclosure relates to medical techniques, in particular to a global irradiation thermotherapy system based on coordinated rotation of microwave and radio frequency, and a global thermotherapy instrument.

### BACKGROUND

Currently, surgery, chemotherapy, and radiotherapy are considered as the most conventional methods for the clinical treatment of cancer. They alone or in combination have good curative effects on the early radical treatment of tumors that have not experienced metastasis, but they are often less effective for the treatment of middle and advanced tumors, and thus fail to prevent the metastasis and spread. Therefore, scientific researchers in the medical field all over the world have been committed to finding and exploring new treatment approaches for a long time. It is recorded in history that the German doctor Busch concerned in 1866 that one of his cancer patients had not received any anti-cancer treatment, but the high fever caused by erysipelas infection accidentally caused the cancer to disappear, and for the first time proposed the thermotherapy theory that high fever can cure cancer. In 1893, Coley published his research on “febrile therapy” in the American Journal of Medicine. He experimentally injected tumor patients with a mixed extract of Streptococcus pyogenes and Pseudomonas aeruginosa (that is erysipelas toxin, also known as Coley toxin) to induce the patient to have a fever of 38.0˜40.2° C., 38 patients with advanced malignant tumors, of which 12 cases completely resolved, 19 cases improved, and the 5-year survival rate reached 60%. In 1918, Robdendury reported that among the collected 166 cases of malignant tumors that resolved spontaneously without any treatment, among them, 72 cases were found to be accompanied by severe infection and high fever. In 1957, Selawry synthesized 450 cases of malignant tumors that had spontaneously resolved through various documents and found that at least 150 of them had a history of malaria and typhoid fever that induced high fever. In the 1980s, Ishihara, a Japanese medical doctor, published “Body Temperature Determines Birth, Old Age, Sickness and Death”, proposing the idea that a temperature exceeding 39° C. can scald cancer cells to death. In 1985, the FDA (U.S. Food and Drug Administration) certified that thermotherapy is the fifth largest tumor treatment after surgery, radiotherapy, chemotherapy, and biological therapy, and pointed out that thermotherapy is a green therapy! Since then, many countries have actively carried out research and adopted various heating methods to verify the theory of thermotherapy. The successive results have all proved that thermotherapy is a rare method for the treatment of cancer, and the development of thermotherapy equipment has a good prospect.

The inventor of the present invention comprehensively believes that in order to realize the radical cure of tumors, clarify the mechanism of thermotherapy treatment, search for global thermotherapy technology, optimize the best temperature range for high fever and blanching, and ensure accurate temperature control have become the four major technical problems.

The inventors have also conducted in-depth research on cancer cells and found that due to the rapid differentiation of genetic mutations, cancer cells grow out of control, and have the ability to proliferate indefinitely. Under normal circumstances, they need to absorb a large amount of nutrients from the outside world and input excessive oxygen. At this time, the stress response is that respiration will rapidly intensify. Due to mutations such as tissue vascular disorder and obstruction due to cancer cell gene mutations, it is difficult to adapt to the excessive demand for oxygen by cancer cells under high fever. As a result, it will cause anaerobic respiratory stress, and a large amount of lactic acid produced by anaerobic accumulation will not have time to efflux, leading to acidification of cancer cell tissues. These acidified substances cause fatal harm to cancer cell tissues: acidic environment will inhibit cancer cell protein and chromosome replication and cause proliferation failure. At this time, cancer cells can continue surviving and not being scalded to death, but no longer differentiated and proliferated, which is equivalent to losing the replication function, and falling into a state of apoptosis. This inactive cancer cell is then eliminated through two ways: one is that one by one will continue to die with the metabolism, secondly, because of its acidic characteristics, it is easier to be recognized and eliminated by immune cells such as lymph. Therefore, the impact of high fever and blanch on cancer cells is an irreversible killing. One or more thermotherapy can achieve a radical cure. In the human body tolerable temperature range (within 47° C.), the higher the temperature, the stronger the anaerobic respiratory stress of cancer cells, and the more thorough the apoptosis caused by the acidification of cancer cell tissues. The high fever over 42° C. will cause cancer cells to die if they are blanched for more than 30 minutes, the mortality rate is more than 90%, but if the temperature exceeds 43° C., the high fever will be more and more harmful to the normal cells of the human body. Therefore, the best temperature range for high-temperature blanching should be 4243° C., and the highest limit should not exceed 43° C. Cancer cells and normal human cells exhibit significant difference at 43° C., specifically, the cancer cells become inactivated while the normal cells are less damaged, which makes this temperature possible for thermotherapy to cure tumors.

In-depth studies have found that although the current various tumor thermotherapy methods have certain feasibility, they also have various problems and shortcomings, which affect the final curative effect to varying degrees, making it difficult for the existing thermotherapy techniques to cure the tumor. In order to facilitate the improvement of the process and the development of more scientific and reasonable thermotherapy technology, we have carried out a systematic analysis and research on various existing thermotherapy methods, found out the crux of the problem, and proposed improvement ideas.

At present, thermotherapy methods are divided into two categories: one is non-equipment thermotherapy methods, which are mostly based on the most primitive cases of measles or malaria infection with high fever to treat cancer, and inject “toxin” into cancer patients to induce infection and fever, so as to burn the cancer cells. These methods have definite results, but the risks are difficult to control and cannot be promoted. The other is equipment-based thermotherapy, which uses equipment to heat to kill cancer cells. According to different heating methods, the equipment-based thermotherapy methods are divided into the following types.

One is the space capsule tumor thermotherapy system, which allows patients to be placed in the space capsule to perform thermotherapy with infrared rays or high-temperature steam. Although this is a global heating idea, because the human body has the function of self-defense, it resists the outside high temperature through sweating to guarantee a constant temperature inside the human body, so infrared or high-temperature steam has a poor effect on the human body, and it is difficult for the deep part of the human body to reach high temperature. This equipment is only effective for shallow tumors that have not metastasized.

The second is a tumor microwave thermotherapy device. Theoretically, the following four characteristics of microwave are very suitable for making a tumor thermotherapy instrument: (1) the heating penetration is good: the inside and outside of the medium can be heated uniformly at the same time; (2) select heating: only for the water-containing tissue generates heat, but almost penetrates through the tissues with low water content such as fat and protein without generating heat; (3) the thermal inertia is small, and it stops at once, which is conducive to precise temperature control; (4) non-ionizing: the effect of microwave on the human body is only physical heating effect, no chemical hazard. However, from the actual application of the N-9001 microwave tumor therapy instrument (915 MHz) developed by Jiangsu Nowan Medical Equipment Co. Ltd. and the UNI-3000 microwave thermotherapy device (2450 MHz) developed by Hunan Youli Medical Technology Co. Ltd., there are some problems with microwave thermotherapy. Technical defects, in-depth research found that the root causes are: first, the microwave penetration depth is not enough, usually no more than 6 cm (including fat thickness), the directional heating coverage is very small, and it is limited to the treatment of superficial tumors that have not metastasized.

The third is the tumor radio frequency thermotherapy device, which is a new type of electromagnetic thermotherapy device based on the research and improvement of microwave thermotherapy technology. Although it has broken through the limitation of the depth of diathermy, the application effect is still not optimistic, and it is difficult to radically cure the spread and metastasis tumor. Through in-depth research on various radio frequency thermotherapy devices such as Jilin Maida NRL-003 (30˜40 MHz difference frequency), Japan FR-8 (8 MHz), American BSD-2000W (60˜120 MHz), etc., it is found that there are two common problems in all these radio frequency thermotherapy devices: firstly, radio frequency heating tends to cause overheating of the superficial fat layer, which must be laminated with a water bag for cooling treatment. Secondly, the current radio frequency thermotherapy device adopts multiple sets of capacitive radio frequency rotary heating mechanisms to directional cross radiation to strengthen the heating and scald the found tumor lesions, and lacks global thermotherapy thinking and radical cure concepts.

Chinese Patent Publication No. 106823152A, titled “A Cancer Therapeutic Apparatus” and Chinese Patent Publication No. 110694178A, titled “A Tumor Horizontal Microwave Global Thermotherapy Device” both disclosed a global treatment device, but due to the insufficient heat penetration depth of a single microwave thermotherapy, even if the rotary heating method is used, only a ring-shaped high temperature layer with the thickness no more than 4 cm can be formed inside the fat layer, and the temperature gradually decreases from the outside to the inside. It can kill non-metastatic tumors located in the superficial layer, and cannot be used for deep tumor treatment. The comparison of the effects of microwave static directional heating and microwave rotary heating is shown in FIG. 2 and FIG. 3.

In addition, field investigations also found that today's human body internal temperature measurement technology has shortcomings. Various thermotherapy devices are not equipped with accurate internal temperature measurement instruments. Thermotherapy cannot accurately control the temperature. For safety reasons, it is forced to lower the temperature at the expense of the effect of thermotherapy. Conservative irradiation also affects the real effect of thermotherapy equipment, so current thermotherapy devices have not achieved the expected results.

At present, there are only two types of temperature measurement that are suitable for anti-electromagnetic interference in the body. One is the optical fiber method, which can accurately measure the temperature of certain positions in the body through invasive implantation. Although it is not subject to electromagnetic interference, it can be used for multi-point temperature measurement in the body. However, invasive implantation not only increases the patient's pain and infection risk, but is also restricted by many factors. It cannot reach any part of the human body (such as the liver, lungs, kidneys and other organs) at will, so it is impossible to obtain the temperature information of all the location in internal parts of the human body. Another type of nuclear magnetic thermal imaging method uses nuclear magnetic thermal technology combined with big data to calculate through software. Although it can obtain the human body temperature information non-invasively, the temperature measurement error of this method is relatively large, which is not conducive to precise temperature control.

In-depth research has found that the temperature measurement errors of nuclear magnetic thermal imaging technology are mostly caused by changes in environmental factors such as voltage, humidity, magnetic field and host operating temperature. The errors caused by changes in these factors have the same deviation characteristics in the same direction. This discovery is based on the use of optical fibers. The temperature measurement technology has found a basis for correcting the temperature measurement error of nuclear magnetic thermal imaging, and is also conducive to the invention of a method for accurate and non-destructive temperature measurement in the body.

Based on the above analysis, it can be seen that there is an urgent need to provide a new type of microwave radio frequency coordinated rotation global radiation thermal therapy system and its device to solve the above-mentioned problem of global thermal therapy under precise temperature control.

### SUMMARY

The technical problem to be solved by the present disclosure is to provide a global irradiation thermotherapy system based on coordinated rotation of microwave and radio frequency, and a global thermotherapy instrument, which utilize microwave and radio frequency rotation irradiation combined with precise temperature measurement technology to realize the whole area of the human body with precise high heat and blanching.

In order to solve the above technical problems, the first technical solution adopted by the present disclosure is to provide a global irradiation thermotherapy system based on coordinated rotation of microwave and radio frequency, which mainly includes an intelligent control unit, a microwave rotary heating mechanism, a capacitive radio frequency rotary heating mechanism, and a temperature measuring mechanism connected to the intelligent control unit;

the capacitive radio frequency rotary heating mechanism is configured to rotationally heat each point in an area of a human body with a lateral radius of 0˜13 cm through the annular phantom to form a circular high-temperature zone, wherein a temperature of the circular high-temperature zone decreases from center to outside, and fat tissues in an outermost layer of the human body has the lowest temperature;

the annular phantom is adapted to the capacitive radio frequency rotary heating mechanism, and comprises a liquid-injection annular capsule, a cylindrical rib frame located at an inner side of an outer surface of the liquid-injection annular capsule, and a hoop located outside two ends of the liquid-injection annular capsule along an axial direction; an outer ring surface of the liquid-injection annular capsule is in rotational contact with a radio frequency electrode plate of the capacitive radio frequency rotary heating mechanism; and the outer ring surface of the liquid-injection annular capsule is made of a flexible insulating material, and is supported by the cylindrical rib frame and the hoop to maintain a cylindrical shape; an inner ring surface of the liquid-injection annular capsule has a soft and elastic structure; when the liquid-injection annular capsule is empty, the inner ring surface of the liquid-injection annular capsule constricts to fit an inner side of the outer ring surface of the liquid-injection annular capsule; and when the liquid-injection annular capsule is filled with a liquid, the inner ring surface of the liquid-injection annular capsule fits and surrounds skins of the human body to play a role as a transition medium between the radio frequency electrode plate and the skins of the human body;

the microwave rotary heating mechanism is configured to form an annular high-temperature layer with a thickness of 2˜4 cm on an inner side of a fat tissue through rotary microwave irradiation, and a temperature of the high-temperature layer decreases from outside to inside;

the temperature measurement mechanism is configured to obtain precise temperature data of several key points in an accessible part of a thermotherapy area of the human body via optical-fiber temperature-measuring technology as a reference system, and to accurately measure a temperature of each point in the thermotherapy area based on combination of nuclear magnetic thermal imaging technology; and

the intelligent control unit is configured to coordinately control the microwave rotary heating mechanism and the capacitive radio frequency rotary heating mechanism to rotationally irradiate the thermotherapy area to perform layered complementary diathermy, and to perform precise temperature control based on real-time temperature measurement of the temperature measuring mechanism to achieve uniform and precise hyperthermia throughout the thermotherapy area.

In an embodiemnt, a microwave frequency of the microwave rotary heating mechanism is 300 MHz˜3000 MHz, and a radio frequency of the capacitive radio frequency rotary heating mechanism is 1 MHz˜300 MHz.

In an embodiment, the temperature measuring mechanism comprises a nuclear magnetic thermal imaging device, an optical fiber temperature measuring device, and a data processing platform;

the nuclear magnetic thermal imaging device is configured to establish a temperature field distribution map library of hot points respectively corresponding to individual temperatures according to nuclear magnetic resonance scanning data, and to obtain a temperature field distribution map of the thermotherapy area of the human body by scanning;

the optical fiber temperature measuring device is configured to obtain the temperatures of several key points in the accessible part of the thermotherapy area as temperature calibration points by using a non-invasive technology; specifically, the optical fiber can be inserted into the stomach and intestine through the esophagus and anus, or the optical fiber can be inserted into the skull through the ear canal and nasal cavity, and other non-invasive ways;

the data processing platform is configured to calculate the temperature field distribution map scanned by the nuclear magnetic thermal imaging device based on the temperature field distribution map library to obtain a temperature scanning data of each hot point in the thermotherapy area, and is configured to calibrate the temperature scanning data of each hot point using temperature data of the temperature calibration points as reference to obtain accurate temperature data of each hot point in the thermotherapy area. In an embodiment, the optical fiber temperature measurement device includes a temperature measuring device comprises a temperature data acquisition unit, a temperature data processing unit and a sensing optical fiber; and a plurality of optical fiber sensors are provided on the sensing optical fiber.

In an embodiment, the cylindrical rib frame consists of two C-shaped structures to facilitate opening and closing of the liquid-injection annular capsule.

In an embodiment, the hoop consists of two C-shaped structures; the hoop is fixedly sleeved on outer sides of two ends of the liquid-injection annular capsule, respectively; and the hoop is configured to be opened and closed together with the liquid-injection annular capsule.

In an embodiment, the inner ring surface of the liquid-injection ring annular capsule has a spliced structure, and is composed of a first flexible layer and a middle elastic layer and a second flexible layer along an axial direction of the liquid-injection annular capsule; and the middle elastic layer is configured to fit and surround the skins of the human body after being filled with the liquid.

In an embodiment, the outer ring surface of the liquid-injection annular capsule is cylindrical, and is configured to be compressed by the radio frequency electrode plate to increase a contact area.

In an embodiment, the liquid injection annular capsule is composed of two C-shaped closed-mouth capsules, which are arranged in an up-and-down manner to surround a body cavity, both ends of the liquid-injection annular capsule along the axial direction are respectively provided with a ring-shaped soft cloth with a contractile inner opening; and the liquid filled in the liquid-injection annular capsule is a non-polar liquid.

In an embodiment, the system also comprises a translational heat-preservation bin, wherein the translational heat-preservation bin is configured to carry the human body in and out of the internal space of the microwave rotary heating mechanism and the capacitive radio frequency rotary heating mechanism.

In an embodiment, the system also includes a rotating mechanism, which is configured to drive a global radiation ring equipped with the capacitive radio frequency rotary heating mechanism or/and the microwave rotary heating mechanism to rotate or brake according to a control program.

In a second aspect, the disclosure provides a temperature measurement method based on the temperature measurement mechanism, comprising:

(S401) scanning hot points using the nuclear magnetic thermal imaging device to obtain thermal imaging data, and establishing a temperature field distribution map library of the hot points respectively corresponding to individual temperatures;

(S402) obtaining the temperature field distribution map of the thermotherapy area of the human body by scanning using the nuclear magnetic thermal imaging device;

(S403) obtaining, by the optical fiber temperature measuring device, temperatures of several key points in the accessible part of the thermotherapy area as temperature calibration points by using a non-invasive technology; and

(S404) calculating the temperature field distribution map obtained in step (S402) based on the temperature field distribution map library obtained in step (S401) to obtain a temperature scanning data of each point in the thermotherapy area; and calibrating the temperature scanning data of each point in the thermotherapy area using temperature data of the temperature calibration points as a reference to obtain accurate temperature data of each point in the thermotherapy area.

In an embodiment, in step S401, the temperature field distribution map library comprises thermal imaging data and temperature value of each hot point, and the thermal imaging data and temperature value of each hot point are in one-to-one correspondence.

In an embodiment, in step S404, the temperature data of the temperature calibration point comprises accurate temperature value and location information; and the temperature field distribution map comprises the thermal imaging information and location information of each point in the thermotherapy area; and the temperature calibration points have corresponding points in the temperature field distribution map.

In a third aspect, the disclosure provides a global thermotherapy instrument based on the microwave radio frequency coordinated rotation global irradiation thermotherapy system, comprising:

a global irradiation unit;

wherein the global irradiation unit comprises a microwave rotary heating mechanism, a capacitive radio frequency rotary heating mechanism, a rotating mechanism and an annular phantom; the microwave rotary heating mechanism and the capacitive radio frequency rotary heating mechanism are arranged on the same global irradiation ring or are respectively arranged on two global irradiation rings; the annular phantom is used to make up for irregularity of the human body to form a regular cylinder, so as to allow the capacitive radio frequency rotary heating mechanism to perform diathermy; the rotating mechanism is configured to drive the global irradiation ring to rotate; and based on combination of microwave rotary heating and capacitive radio frequency rotary heating, the global thermotherapy instrument achieves layered complementary diathermy, so as to achieve accurate and uniform hyperthermia in the entire thermotherapy area.

In an embodiment, the global thermotherapy apparatus further comprises a temperature measuring unit for real-time non-invasive temperature measurement of an object, the temperature measuring unit comprises an optical fiber temperature measuring device, a nuclear magnetic thermal imaging device and a data processing platform.

In an embodiment, the global thermotherapy instrument further comprises a translational heat-preservation bin, the translational heat-preservation bin comprises a bin base, and a head holder; and the bin body passes through the nuclear magnetic thermal imaging device and the global irradiation unit in sequence to be supported on the head holder.

In an embodiment, the nuclear magnetic thermal imaging device and the global irradiation unit are together arranged on a guide rail and are capable of sliding back and forth along the guide rail.

In an embodiment, a microwave frequency of the microwave rotary heating mechanism is 300 MHz˜3000 MHz.

In an embodiment, a radio frequency of the capacitive radio frequency rotary heating mechanism is 1 MHz˜300 MHz.

Compared to the prior art, the present disclosure has the following beneficial effects.

(1) The present disclosure uses microwave combined with capacitive radio frequency rotary irradiation to uniformly heat both internal and external. It has a global hot-scalding thinking, fully simulating the human body's high fever and killing cancer cells, and achieves accurate temperature measurement through nuclear magnetic temperature measurement with the help of optical fiber technology, ensuring accurate control of high fever and blanching, stable and controllable heating, and patients can also withstand systemic and local treatments, with good therapeutic effects, no damage to healthy tissues, and no adverse side effects.

(2) The present disclosure takes advantage of the deep heating of radio frequency and adopts the rotary heating method to strengthen the scanning and heating of each point in the human body with a lateral depth of more than 6 cm, avoiding the problem of overheating of the surface fat layer caused by radio frequency radiation, and enabling the deep layer to be focused to achieve high temperature. At the same time, the use of the annulus phantom matched with the radio frequency rotary heating mechanism solves the problem of irregular body cavity and the difficulty of tightly fitting the radio frequency plate during rotation and irradiation, which affects the irradiation effect. After the annulus phantom being worn and used by the patients, the radio frequency electrode plate can not only achieve the technical effect of rotating and irradiating the outer circumference of the annulus phantom with the cooperation of lubricant, but also solve the requirement of the electrode plate and the outer peripheral surface of the annulus phantom to expand the close contact area. Resulting in reducing the difficulty of radio frequency rotary irradiation control, and improving the effect of radio frequency and microwave coordinated rotary irradiation.

(3) The present disclosure uses the feature of microwave selective heating to avoid the superficial fat layer, to ensure that the fat will not overheat, and to compensate the defect that the 2˜6 cm middle layer is not enough due to the insufficient heat of the radio frequency rotary radiation to realize the overall uniform heating.

(4) The present disclosure combines the nuclear magnetic resonance thermal imaging technology with the optical fiber temperature measurement technology, which not only realizes the non-invasive measurement of the wide-area temperature in the body, greatly reduces the pain of the patient, but also realizes accurate temperature measurement and realizes the precise control of thermotherapy temperature, ensures the safety and efficiency of thermotherapy.

(5) The system and the global thermotherapy device of the present disclosure adopt pure physical precision temperature control thermotherapy, which is safe, convenient and efficient, and is applied in the field of tumor treatment, which can cure various solid tumors and keep normal tissues and organs intact. It has the characteristics of short time, less pain and low cost for tumor treatment, and has a wide range of indications.

### BRIEF DESCRIPTION OF THE DRAWINGS

FIG. 1 is a structural block diagram of the microwave radio frequency coordinated rotation global radiation thermotherapy system of the present disclosure;

FIG. 2 is a schematic diagram of the structure of the annulus phantom;

FIG. 3 is a schematic diagram of the state of use of the annular phantom, which uses a cross section to show the matching relationship between the chest cavity and the liquid-injection annular capsule;

FIG. 4 is a structural schematic diagram according to the longitudinal section of the annular phantom;

FIG. 5 is a schematic diagram of the structure of the cylindrical rib frame according to the annular phantom;

FIG. 6 is a schematic diagram showing the cooperation between the annulus phantom and the radio frequency electrode plate;

FIG. 7 is a schematic diagram of the effect of microwave static directional heating;

FIG. 8 is a schematic diagram of the effect of microwave rotation heating;

FIG. 9 is a schematic diagram of radio frequency static unidirectional heating effect;

FIG. 10 is a schematic diagram of the effect of static two-way radio frequency heating;

FIG. 11 is a schematic diagram of the effect of radio frequency rotary heating;

FIG. 12 is a schematic diagram of the effect of combined microwave-radio frequency rotary heating at the initial stage;

FIG. 13 is a schematic diagram of the constant temperature effect after the microwave radio frequency rotation cooperates with the heating to slowly supplement the heat;

FIG. 14 is a schematic flow chart of the global irradiation thermotherapy method according to an embodiment of the disclosure;

FIG. 15 is a schematic diagram of the structure of the global thermotherapy instrument according to an embodiment of the disclosure;

FIG. 16 is a schematic diagram of the structure of the radio frequency microwave superimposed radiation ring of the thermotherapy apparatus according to an embodiment of the present disclosure;

FIG. 17 is a schematic diagram of setting two global irradiation rings in the thermotherapy instrument according to an embodiment of the present disclosure; and

FIG. 18 is a schematic diagram of the temperature measuring unit and the global irradiation unit arranged on the guide rail according to an embodiment of the present disclosure.

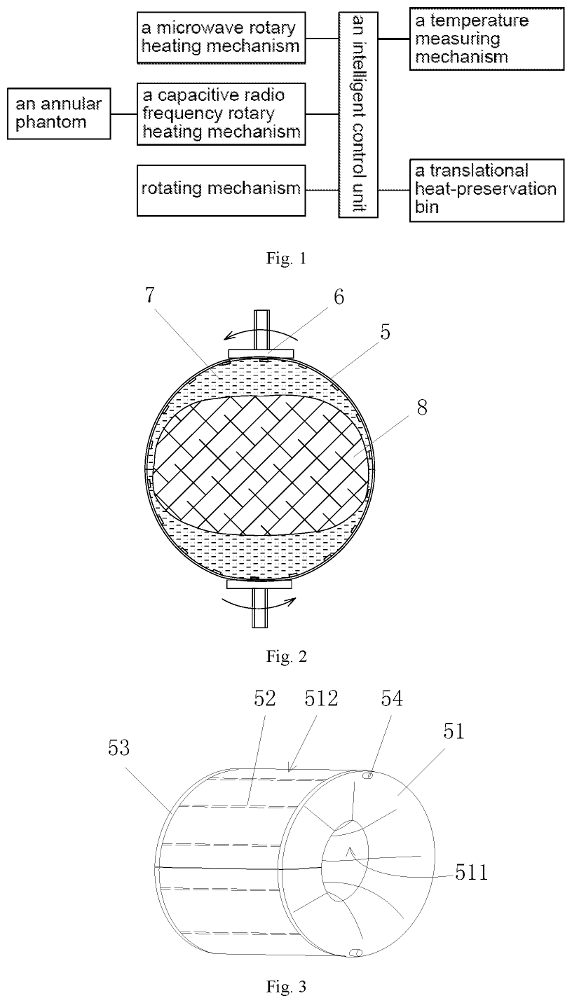

In the drawings: 1. translational heat-preservation bin, 11. bin body, 111. head protection bin, 112. trunk support bin, 12. bin seat, 13. head holder, 2. temperature measuring mechanism, 3. global irradiation unit, 31. holder, 32. rotating drum, 33. global irradiation ring, 34. drive roller, 35. support roller, 36. capacitor plate, 37. microwave magnetron, 4. intelligent control unit , 5. annular phantom, 51. injection annular capsule, 511. outer ring surface, 512. inner ring surface, 5121. front flexible surface layer, 5122. middle elastic surface layer, 5123. rear flexible surface layer, 52. tube body rib frame, 521. hard rib, 522. half ring body, 53. hoop, 54. liquid-injection nozzle, 55. annular soft cloth, 56. wall surface, 6. radio frequency plate, 7. liquid, and 8. body cavity .

### DETAILED DESCRIPTION OF EMBODIMENTS

The preferred embodiments of the present disclosure will be described in detail below with reference to the accompanying drawings to make the advantages and features of the present disclosure more easily understood by those skilled in the art, so as to make the protection scope of the present disclosure clear and definite.

Referring to an embodiment shown in FIG. 1, a global irradiation thermotherapy system based on coordinated rotation of microwave and radio frequency is provided, which mainly includes an intelligent control unit 4, a microwave rotary heating mechanism, a capacitive radio frequency rotary heating mechanism, a temperature measuring mechanism 2, a translational heat-preservation bin 1, and a rotating mechanism connected to the intelligent control unit 4.

The capacitive radio frequency rotary heating mechanism uses an annular phantom to heat up each point of the human body with a lateral radius of 0˜13 cm to form a circular high-temperature zone. The temperature gradually decreases from the center to the outside, and the temperature of the outermost fat tissue of the human body is the lowest. The microwave rotary heating mechanism utilizes microwave rotary irradiation to form an annular high temperature layer with a thickness of 2˜4 cm on the inner side of the fat, and the temperature gradually decreases from the outside to the inside. The capacitive radio frequency rotary heating mechanism and the microwave rotary heating mechanism are arranged on one or two global irradiation rings 33, and the rotating mechanism is used to drive the global irradiation ring 33 to rotate or brake according to a control program. The temperature measurement mechanism 2 uses the internal temperature data of the human body obtained by optical fiber temperature measurement technology as a reference frame, and realizes non-invasive accurate temperature measurement in vivo through the cooperation of nuclear magnetic thermal imaging technology. The translational heat-preservation bin 1 is used to carry the human body in and out of the internal space of the microwave rotary heating mechanism and the capacitive radio frequency rotary heating mechanism. The intelligent control unit 4 is used to coordinate the microwave rotary heating mechanism and the capacitive radio frequency rotary heating mechanism to rotate and irradiate heating around the human body thermotherapy area to perform deep and shallow layered complementary diathermy, and at the same time combine with the real-time accurate measurement of the temperature measurement mechanism 2 to control the temperature precisely, and achieve uniform and precise high-heat blanching throughout the entire area.

Furthermore, the temperature measurement mechanism 2 includes a nuclear magnetic thermal imaging device, an optical fiber temperature measurement device, and a data processing platform. The nuclear magnetic thermal imaging device is used for establishing a temperature field distribution map library of each temperature corresponding to the hot spot according to the scanning data of the nuclear magnetic thermal imaging device, and scanning to obtain the temperature field distribution map of the thermal treatment area of the human body; the optical fiber temperature measuring device uses non-invasive technology to obtain the temperature of several key points in the accessible part of the human body as the temperature calibration point; the data processing platform is used to combine the temperature field distribution map library scanned by the nuclear magnetic thermal imaging device to calculate the temperature field distribution map scanned by the nuclear magnetic thermal imaging device to obtain the scanning temperature data of each hot spot in the human body thermotherapy area, and according to the accurate temperature data of the temperature calibration point as a reference, error correction is performed on the scanned temperature data to obtain accurate temperature data of each hot spot in the thermotherapy area.

In an embodiment, the optical fiber temperature measurement device is a real-time, on-line, continuous point optical fiber temperature measurement system, which includes a temperature information acquisition unit, a temperature information processing unit, and a sensing fiber. The sensing fiber is a kind of anti-electromagnetic interference point temperature sensor, on which there are several optical fiber sensors.

The annular phantom 5 matched with the radio frequency rotary heating mechanism includes a liquid-injection annular capsule 51, a cylindrical rib frame 52 located inside the liquid-injection annular capsule 51, and hoops 53 located at the left and right ends of the liquid-injection annular capsule 51 in the axial direction.

The outer ring surface 511 of the liquid-injection annular capsule 51 is a cylindrical surface in contact with the radio frequency electrode plate 6. The cylindrical surface is made of a flexible insulating material and is supported by the cylindrical rib frame 52 to maintain its shape. The inner ring surface 512 is a soft and elastic structure, which is tightly attached to the outer ring surface 511 when it is not filled with solution, and is attached to the surface surrounding the body cavity 2 when the liquid 7 is filled, as a transition between the radio frequency electrode plate and the skin of the human body. The filling liquid 7 is a non-polar fluid substance, a liquid, such as carbon tetrachloride, which is injected/filled by a liquid injection nozzle 54. The filling quantity of the liquid injection nozzle is one or more, for example, the liquid-injection nozzles 54 are arranged on the upper and lower sides on the sides of the ring phantom 5 respectively.

In one embodiment, the liquid injection annular capsule 51 is composed of two C-shaped closed capsules, and the two C-shaped closed capsules are arranged up and down around the body cavity, so that the body cavity is suspended in the annular phantom 5.

In one embodiment, the cylindrical rib skeleton 52 has two C-shaped half-body structures, which facilitates the opening and closing of the liquid-injection annular capsule 51. Correspondingly, the hoop 53 has two C-shaped half-body structures, which can be opened and closed to facilitate wearing the liquid-injection annular capsule 51 on the body.

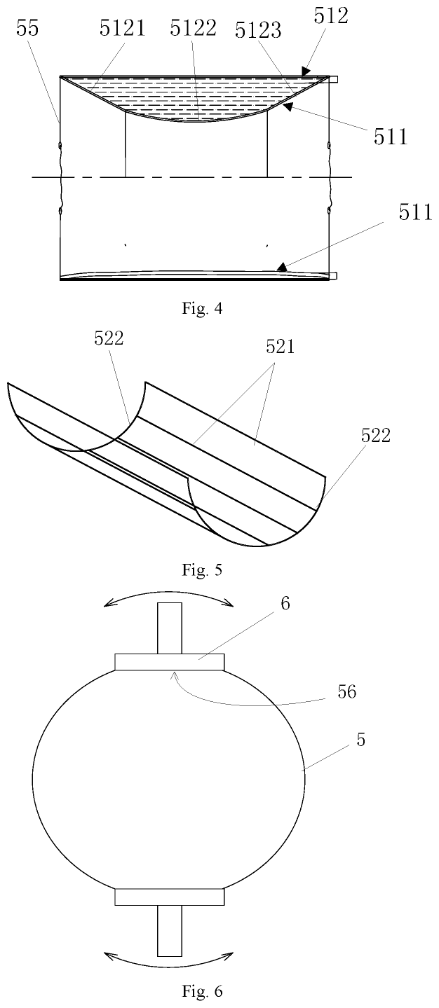

Preferably, as shown in FIG. 4, the half-body structure shown by the cylindrical rib frame is composed of a number of rigid ribs 521 arranged at intervals along the circumference of the cylindrical surface and half-rings 522 at both ends of the cylindrical ribs. The body joint can be opened and closed, allowing the body to enter the liquid-injection annular capsule 51 when opened, and maintaining a cylindrical shape when closed.

In one embodiment, the hoop 53 is located at the two ends of the liquid-injection annular capsule 51 and is combined with the two end rings of the cylindrical rib frame 52, for example, by bonding or clamping.

In one embodiment, as shown in FIG. 3, the inner ring surface 512 of the closed capsule body is a spliced structure, which can be divided into a front flexible surface layer 5121, a middle elastic surface layer 5122, and a rear flexible surface layer 5123 along its axial direction, when there is no liquid, the C-type closed capsule has no liquid type, the inner ring surface 512 shrinks close to the outer wall. When filled with liquid, the front and back flexible surface layers 5121 open, and the middle elastic surface layer 5122 elastically stretches/shrinks to the desired circumference to fit the shape of the body skin. The elastic surface layer can be selected from rubber, latex and other materials, and the front flexible surface layer 5121 and the rear flexible surface layer 5123 can be selected from rubber, waterproof cloth and other materials.

In one embodiment, the liquid-injection annular capsule 51 has an annular soft cloth 55 with a retractable inner opening at both axial ends.

In the embodiment shown in FIG. 1, the appearance of the ring phantom is a regular cylinder with the same semi-warp, which is convenient for the radio frequency plate 6 to rotate around. The annular phantom 5 simulates the body tissue here to solve the problem that the body cavity 8 is not a regular cylinder. It's a problem that the radio frequency polar plate 6 is difficult to fit tightly when rotating and irradiating, which affects the irradiating effect.

As shown in FIG. 5, when the electrode plate 6 is attached to the wall surface 56 of the outer ring surface 511, under appropriate pressure, the wall surface 56 is indented to achieve the radio frequency electrode plate 6 and the side wall tightly fitting, keeping the contact surface to the maximum. Due to the structure of the cylindrical rib frame 52, the wall surface 56 is allowed to be partially recessed, and the wall surface 56 is coated with lubricating oil to allow the radio frequency electrode plate 6 to continuously rotate and irradiate around the side wall of the annular phantom 5.

In a modified embodiment, the outer surface of the annular phantom 5 is oblong, and the radio frequency electrode plate 6 rotates around the outer surface of the oblong circle, so that the radio frequency electrode plate 6 can smoothly rotate along its outer peripheral contour. The annular phantom 5 simulates the body skin of the thin to transform the body skin with an irregular outer contour into a regular body skin, preventing the problem that the radio frequency electrode 6 cannot fit the body skin.

In a modified embodiment, the outer surface of the annular phantom 5 is an ellipse and other smooth shapes.

A microwave radio frequency coordinated rotation global radiation heat therapy method includes the following steps:

Use the intelligent control unit 4 to cooperate with the microwave rotary heating mechanism and the capacitive radio frequency rotary heating mechanism to rotate and irradiate heating around the human body thermotherapy area, and perform deep and shallow layered complementary diathermy to achieve uniform heating of all tissues in the human body thermotherapy area; the temperature measuring mechanism 2 uses the optical fiber technology to obtain the internal temperature data of the human body as the reference frame, and uses the nuclear magnetic thermal imaging technology to perform non-invasive real-time and accurate temperature measurement in the body to achieve precise temperature control and achieve uniform and accurate high heat blanching throughout the area.

The specific steps of the method will be described below in conjunction with specific embodiments and FIG. 14.

### Embodiment 1

(S1) The intelligent control unit 4 uses the microwave rotary heating mechanism and the capacitive radio frequency rotary heating mechanism to rotate and irradiate the human body thermotherapy area in cooperation to perform deep and shallow layered complementary diathermy, and set and adjust the microwave and radio frequency and power according to the tolerance of the human body, guide the translational heat-preservation bin 1 with rotating radiation heating to move back and forth, so that the thermotherapy area of the human body can be heated quickly and generally evenly to 40˜41° C.

In this embodiment, the heating method of microwave combined with capacitive radio frequency rotary irradiation adopts a global irradiation ring 33 for global heating of the human body membrane. The global irradiation ring 33 is provided with one or more pairs of capacitor plates 36 and one or more microwave magnetrons 37 are arranged at intervals in the circumferential direction. One pair or more pairs of capacitor plates 36 are used for radio frequency heat transmission, and the frequency can be selected in the range of 1 MHz˜300 MHz. One or more microwave magnetrons 37 are used for microwave heating, and the frequency can be selected from 300 MHz to 3000 MHz. The capacitor plate 36 is set individually, and the power is not less than 800 W for heating, such as 1000 W. If there are multiple sets, the total power is not less than 800 W for heating. For example, two are set, one of which is 400 W and the other is 500 W. The microwave magnetron 37 is set individually, and the power is not less than 600 W for heating, such as 700 W. If there are multiple microwave magnetrons 37, the total power is not less than 600 W, for example, two are set, one of which is 400 W and the other is 300 W.

Utilizing the advantages of the deep radio frequency heating to uniformly heat the points with a radius of 0˜13 cm, and the radio frequency heating is rotary heating to avoid the overheating of the surface fat layer caused by radio frequency radiation, and it can focus the deep layer to high temperature, and use the characteristic of microwave selective heating, use rotary heating to avoid the superficial fat layer to ensure that the fat will not overheat. For the defect of insufficient heat due to radio frequency rotary radiation in the middle ring zone with a depth of 2˜6 cm, supplementary heating is performed to achieve overall uniform heating. The radio frequency should be 1 MHz˜300 MHz electromagnetic waves, and the microwave should be microwave medium and low-frequency electromagnetic waves of 300 MHz˜3000 MHz.

The following describes the global heating mechanism of the present invention with a set of human body membrane heating effect diagrams in conjunction with this embodiment.

A microwave magnetron 37 adopts 900 MHz, the first set power is 700 W (adjustable), a pair of capacitor plates 36 adopts 40 MHz, the second set power is 1000 W (adjustable), a human body phantom with a radius of 13 cm instead of a real human body section, where the outermost layer is a fat layer with a thickness of 2 cm, the middle layer of 2˜6 cm is the muscle layer, and the other tissue layer is 6˜13 cm. Place it in a 37.3° C. environment, fiber optic sensors are arranged to measure temperature from the center to the edge with different depths.

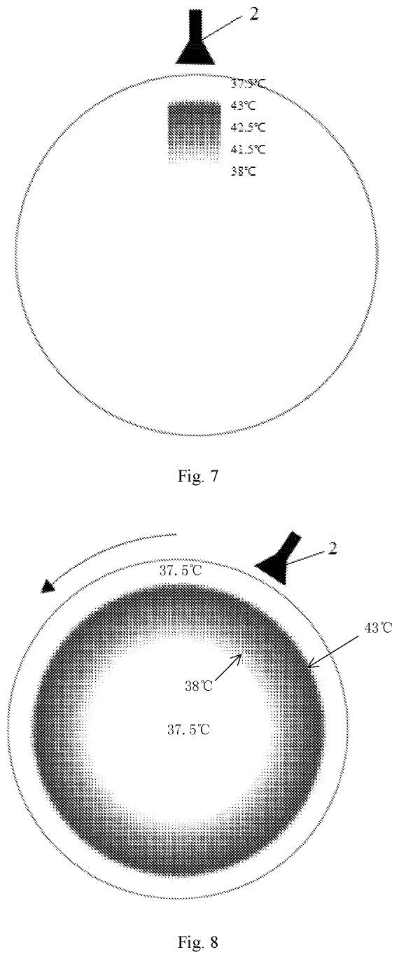

As shown in FIG. 7, the irradiated area facing the microwave magnetron 37 is the local thermotherapy area. The skin temperature outside the fat layer is used as the temperature measurement base point. When the muscle layer inside the fat layer is heated to 43° C., accurate temperature measurement by nuclear magnetic temperature measurement with optical fiber temperature measuring technology, the temperature rise is as follows: the skin temperature is 37.3° C., the fat layer is about 2 cm thick, not easy to heat, the temperature is about 38° C., the temperature of the muscle layer inside the fat layer, it decreases as the depth increases, from 43° C. to 41.5° C. . When the heating depth reaches more than 6 cm, the temperature drops sharply to below 38° C. This part of the temperature rise is related to heat conduction.

As shown in FIG. 8, the irradiation area facing the microwave magnetron 37 rotating and irradiating is an annular area. A 4 cm-thick annular high-temperature layer is formed in the inner layer of fat. Take the skin temperature outside the fat layer as the temperature measurement base point, when the muscle layer on the inner side of the fat layer is heated to 43° C., the temperature is accurately measured by nuclear magnetic temperature measurement and optical fiber temperature measurement technology. The temperature measurement result is that the fat layer is about 2 cm still difficult to heat, and the temperature is about 37.5° C. The temperature of the inner muscle layer decreases from 43° C. to 41° C. with the depth increasing, when the heating depth exceeds 6 cm, the temperature drops below 39° C.

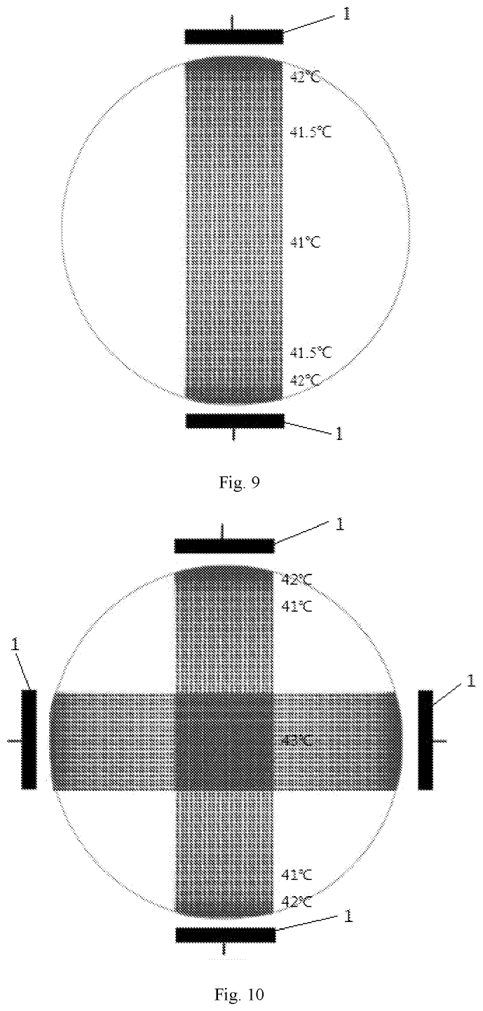

As shown in FIG. 9, the radius of the heating circle section of the body membrane is 13 cm. The belt-shaped area where a pair of capacitor plates 36 facing each other and the body membrane passed is the effective heating area. The fat layer dissipates slowly. Take the skin temperature outside the fat layer as the temperature measurement base point, accurate temperature measurement is carried out through nuclear magnetic temperature measurement and optical fiber temperature measurement technology. The temperature measurement result is: when the temperature of the fat layer is heated to 42° C., the temperature of the muscle layer on the inner side of the fat layer decreases as the depth increases. The temperature decreased from 42° C. to 41° C. from the fat layer to the center of the section.

As shown in FIG. 10, the cross-belt-shaped area where the two pairs of capacitor plates 36 face each other to the body membrane is the effective heating area. The fat layer dissipates slowly. Take the skin temperature outside the fat layer as the temperature measurement base point, accurate temperature measurement by nuclear magnetic temperature measurement with optical fiber temperature measurement technology, the temperature measurement result is: when the temperature of the fat layer is heated to 42° C., the temperature of the muscle layer on the inner side of the fat layer decreases with the increase in depth, from 42° C. to 41.5° C., but the temperature of the cross belt in the central overlapping area rose to 43° C.

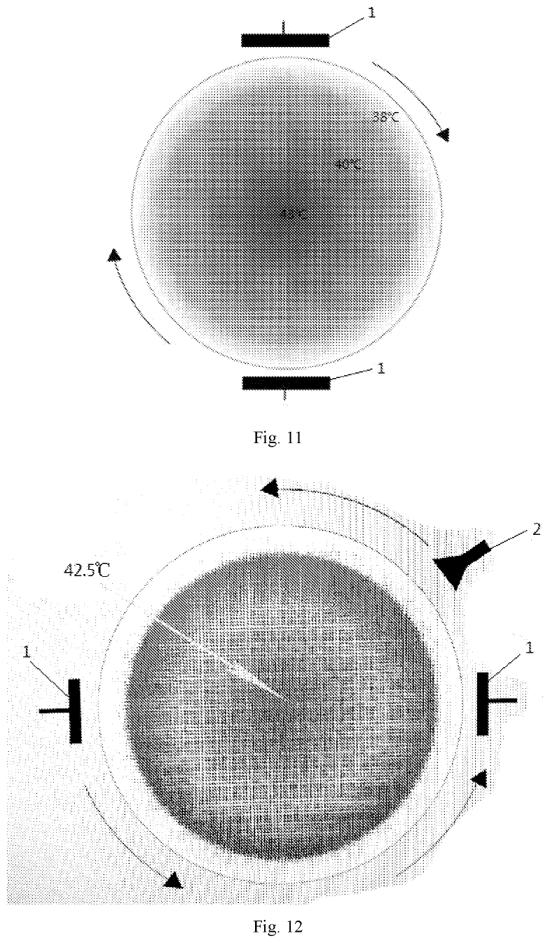

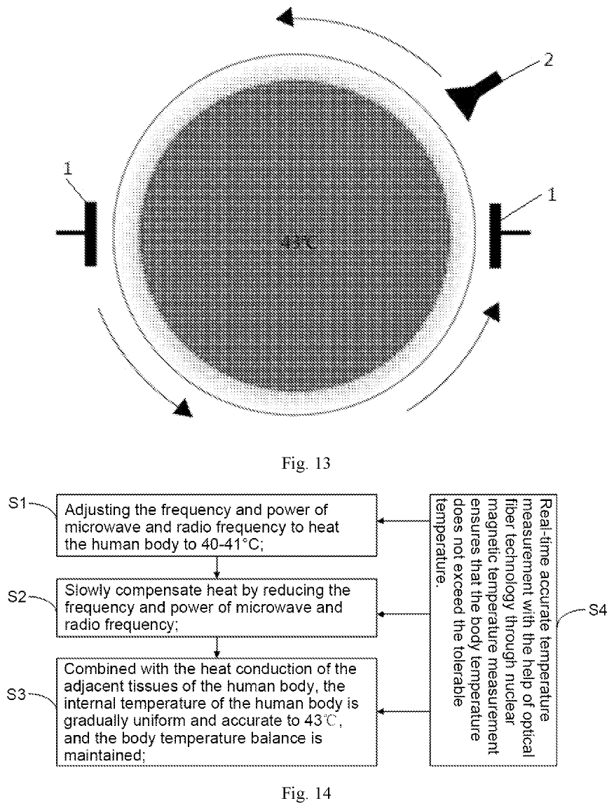

As shown in FIG. 11, when a pair of capacitive pole plates 36 rotate and heat the body membrane alone, the irradiation area facing the entire heating circle section with a radius of 13 cm, with the central temperature as the base point of temperature measurement, accurate temperature measurement by nuclear magnetic temperature measurement with optical fiber temperature measurement technology, the temperature measurement result is: when the central area is heated to 43° C., the temperature of the heating circle section increases with the increase in depth, from the central area to the fat layer, it drops from 43° C. to 39° C. The problem of overheating of the fat layer in the state is improved by rotary heating and the fat layer becomes a relatively low-heat area.

FIG. 12 shows the effect diagram after the microwave rotary irradiation and the radio frequency rotary irradiation are synchronized or superimposed on heating, that is, the superimposed diagrams of FIG. 8 and FIG. 11. After synchronizing or superimposing the heat, precise temperature measurement is carried out by nuclear magnetic temperature measurement and optical fiber temperature measurement technology. When the temperature in the central area reaches about 42.5° C., the fat layer is about 2 cm still in the low temperature area, about 38° C., and the temperature of each tissue of the inner side of the fat layer is maintained at a range of 40˜42° C., showing a state of uneven temperature distribution.

FIG. 11 shows a heating effect diagram after the global radiation ring 33 continues to slowly supplement heat and conduct heat. The entire heating circle section is slowly supplemented by reducing the power of microwave and radio frequency. After a period of slow supplementation, the temperature is gradually increased while the temperature difference between the tissues is eliminated, and the temperature is slowly increased after a certain period of time. The temperature is finally uniform and maintain a constant temperature after reaching 43° C. for certain time to consolidate the effect of thermotherapy.

S2: The intelligent control unit 4 slows down the heating speed by adjusting the power of the microwave rotary heating mechanism and the capacitive radio frequency rotary heating mechanism to supplement the heat of the body membrane thermotherapy area, and it takes no less than 10 minutes for each temperature rise of 1° C.;

In this embodiment, the pair of capacitor plates 36 and the microwave magnetron 37 are slowly supplemented with heat at the third set power and the fourth set power, for example, 100 W.

S3: After the temperature of the body membrane thermotherapy area rises to 42° C., the intelligent control unit 4 further controls to slow down the heating speed, combined with the heat conduction between adjacent tissues of the body membrane, so that the internal temperature of the body membrane is gradually uniform and accurate to 43° C. and maintained the body temperature balance; the intelligent control unit 4 maintains a relative constant temperature in the thermotherapy area for 30˜60 minutes based on the real-time temperature measurement data, under the premise of ensuring that the human body temperature resistance limit is not exceeded;

S4: In the above steps, the internal temperature data of the body membrane obtained with the help of optical fiber temperature measurement technology is the reference system, and the non-invasive in the body precise temperature measurement is achieved through the use of nuclear magnetic thermal imaging technology. The temperature measurement data is transmitted to the intelligent control unit 4 to accurately control the body membrane heat in real time. The temperature of the treatment area does not exceed the tolerance limit. Specifically, the step (S4) is performed through the following steps.

S401: Use the nuclear magnetic thermal imaging device to scan each temperature hot point in a certain temperature range (20˜80° C.), and establish a temperature field distribution map library for each temperature corresponding to the hot point.

The temperature field distribution map library must be composed of an unlimited number of continuous hot point thermal imaging data within a set interval. In actual operation, according to the accuracy requirements, a large number of hot point thermal imaging data statistics can be used to generate a thermal imaging information data map library composed of discontinuous hot spots, as a standard reference system, ensures that the temperature distribution map obtained by each nuclear magnetic thermal imaging device scans has no excessive deviation.

Further, in the temperature field distribution map library, the thermal imaging map data information has a one-to-one correspondence with its temperature value, which can be represented by two-dimensional coordinates.

S402: Obtain the temperature field distribution map of each hot spot in the patient's thermotherapy area (the composition of important hot spots can be selected according to the accuracy requirements) through the scanning of the nuclear magnetic thermal imaging device.

The temperature field distribution map includes thermal imaging information and location information of each hot point in the thermotherapy area (important hot points can be selected according to the accuracy requirements). The image information is a computer language containing temperature information, and the location information can use the form of three-dimensional coordinates.

S403: Use the optical fiber temperature measuring device to use non-invasive technology to obtain the temperature of several key points in the accessible part of the human body thermotherapy area as the temperature calibration point.

Further, the sensing optical fiber is non-invasively guided into the gastrointestinal tract, trachea, bladder, uterus, ear chamber and other tissue cavities in the patient's treatment area from the anus, mouth and nose, urethra, or ear canal, so as to avoid direct minimally invasive surgery of the human body and other forms of temperature measurement fiber into the human body to cause trauma.

Further, the temperature calibration point and its temperature reference include accurate temperature data and location information. The temperature calibration points obtained by the sensing optical fiber have corresponding points in the temperature distribution map, and the changes of the temperature values of both of them often lead to the same error in the same direction due to the change of objective factors.

S404: Combining the temperature field distribution map library obtained by scanning in step S401, calculate the temperature field distribution map obtained in step S402 to obtain the scanning temperature data of each hot spot in the thermotherapy area, and according to the temperature data of the temperature calibration point as the reference standard, the calculated scanning temperature data of each point is used for error correction, and the accurate temperature data of each hot spot in the thermotherapy area is obtained.

For example, if the precise temperature of point A on the sensing fiber measured by the optical fiber temperature measuring mechanism 2 is assumed to be 40° C., the position of the magnetic resonance scan of point A in the temperature field distribution map is point A′, which is compared with the temperature field distribution map library. The scanning temperature is 39.4° C., and the error correction value of the two is +0.6° C. If the position in the temperature field distribution map is point B′, the corresponding scanning temperature is 39.9° C., then the accurate temperature after error correction at this point is 40.5° C.

### Embodiment 2

In this embodiment, the heating method of microwave combined with capacitive radio frequency rotary irradiation still uses the global irradiation ring 33 to heat the human body. The global irradiation ring 33 is provided with a pair of capacitor plates 36 and a pair of capacitor plates 36. Microwave magnetrons 37 are arranged at intervals in the circumferential direction. A pair of capacitor plates 36 is used for radio frequency heat transmission, and the frequency can be selected in the range of 5 MHz˜200 MHz. The microwave magnetron 37 is used for microwave heating, and its frequency can be selected from 300 MHz to 3000 MHz. In step S101, the two pairs of capacitor plates 36 are heated with the first and second set powers, such as 800 W, 300 W, and the two microwave magnetrons 37 are heated with the third and fourth set powers, such as 500 W, 200 W, and the temperature of center area and the muscle layer under the fat layer are the limit temperature points for temperature calibration. In step S102, the two pairs of capacitor plates 36 and the two microwave magnetrons 37 are both set to a low power, such as 80 W, to slowly supplement heat.

Based on the above heating parameters, steps S1˜S4 in Embodiment 1 are performed to implement the global heating of the body. As a consequence, not only the body surface, but also the deep organs can be accurately and easily heated, realizing accurate high heat and blanching throughout the body. The content of steps S1˜S4 will not be repeated here.

In the above global heating method, the heating parameters need to be selected by the physician according to human body constitution.

### Embodiment 3

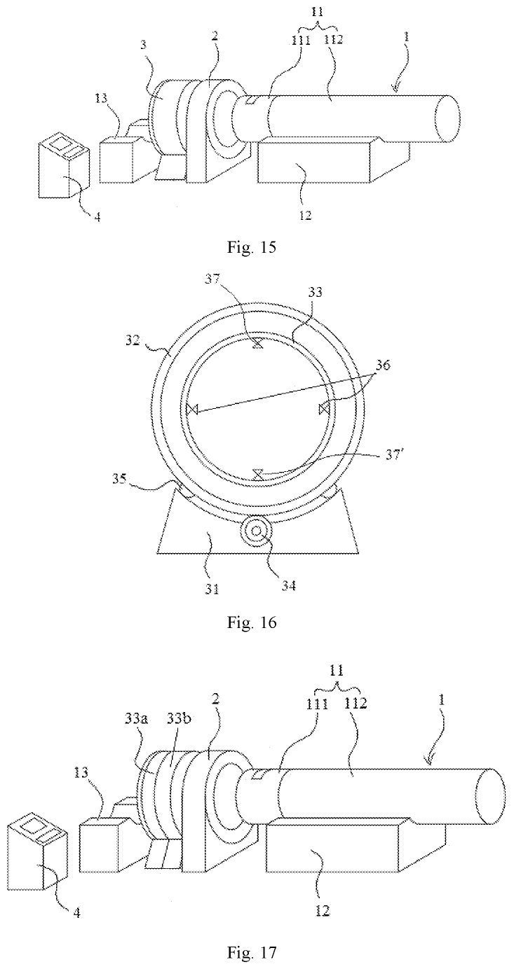

The present disclosure also provides a microwave radio frequency global irradiation thermotherapy instrument, as shown in FIG. 15, including a translational heat-preservation bin 1, a temperature measuring mechanism 2, a global irradiation unit 3, and an intelligent control unit 4.

The translational heat-preservation bin 1 includes a bin body 11, a bin holder 12 and a head holder 13. The bin body 11 includes a head protection bin 111 and a trunk support bin 112.

The bin body 11 is horizontally and slidably supported on the bin holder 12 and is driven by a drive mechanism provided in the bin holder 12. After the bin body 11 passes through the temperature measuring mechanism 2 and the global irradiation ring 3 in turn, the head protection bin 112 is supported on the head holder 13.

The temperature measurement mechanism 2 is used for real-time measurement of the temperature of any part of the human body section, and includes a nuclear magnetic thermal imaging device for collecting a temperature field distribution map of the human body section and an optical fiber temperature measurement device for temperature measurement at a temperature calibration point.

The temperature measurement method of the temperature field distribution map is as follows: first, the sensor fiber of the fiber optic temperature measuring device is introduced into the human esophagus, stomach or intestine through the mouth, nose or anus in a non-invasive manner. The sensor fiber can accurately measure the point temperature value of the path, take these points on the sensing fiber as temperature calibration points, and then scan the cross-section of the human body by the nuclear magnetic thermal imaging device to obtain the temperature field distribution map. Since the temperature calibration points are also in the thermal imaging scanning range, using the temperature of these calibration points as a reference, and the temperature value of each point in the thermal imaging range can be obtained in real time by comparing the temperature difference according to the temperature field distribution map.

As shown in FIG. 16, the global irradiation unit 3 includes a global irradiation ring 33, a microwave rotary heating mechanism, a capacitive radio frequency rotary heating mechanism, a rotating mechanism, and an annular phantom 5 (not shown in FIG. 16). The rotating mechanism includes a holder 31, a rotating drum 32, a driving roller 34, and a plurality of supporting rollers 35. The rotating drum 32 is placed on the holder 31 and supported by a number of supporting rollers 34, and is driven to rotate by the driving roller 33 to drive the global irradiation ring 33 to rotate.

The global irradiation ring 33 is provided with one or more microwave magnetrons 37 of the microwave rotary heating mechanism and one or more pairs of capacitive plates 36 of the capacitive radio frequency rotary heating mechanism. The capacitive plate 36 and the microwave magnetron 37 are arranged at intervals in the circumferential direction of the torus.

The microwave magnetron 37 is used for microwave heating, and its frequency can be selected in the range of 300 MHz˜3000 MHz. The other microwave magnetron 37′ has the same frequency range and can choose microwave devices with different powers. The capacitor plate 36 is used for radio frequency diathermy, and its frequency can be selected in the range of 1 MHz˜300 MHz, and the high frequency band realizes non-skin contact heating. The other capacitor plate 36′ can be eccentrically arranged to solve the problem of weak heat accumulation in the middle layer.

The rotating mechanism is used to drive the global irradiation ring 33 to rotate or brake according to the control program.

The intelligent control unit 4 is used to control the rotation speed of the global radiation ring 33, the radio frequency and power of the capacitor plate 36 (36′), the microwave frequency and power of the microwave magnetron 37 (37′), and the feed location of the bin body 12.

In the translational heat-preservation bin 1 of the thermotherapy instrument, the base 31 drives the bin body 11 to feed, and the feeding position is controllable. The nuclear magnetic thermal imaging device can collect the temperature field distribution map of the human body section containing the optical fiber temperature probe, using the temperature measured by the optical fiber temperature probe in real time is used to display the temperature of any part of the cross section of the human body after temperature calibration of the temperature field distribution map. The global irradiation unit 3 has five modes: microwave static mode, microwave rotation mode, radio frequency static mode, radio frequency rotation mode, and microwave radio frequency combined mode for physicians to choose from.

This thermotherapy instrument uses the advantages of deep radio frequency heating to uniformly heat all points in the treatment area, and the radio frequency heating is rotary heating to avoid the overheating of the surface fat layer caused by radio frequency radiation, and it can focus the deep layer to high temperature, and use the feature of microwave selection heating to avoid the shallow fat layer to ensure that the fat will not overheat. The middle layer high temperature ring is formed by rotating radiation, and the middle layer inside the fat is heated by insufficient heat due to the radio frequency rotary radiation, and the heat is supplemented to achieve uniform heating. The frequency of radio frequency should be 1˜300 MHz, and the frequency of microwave should be 300˜3000 MHz. In addition, the capacitive radio frequency rotary heating mechanism utilizes an annular phantom matched with capacitive radio frequency rotary irradiation to compensate for irregular defects of the human body to form a regular cylinder, which facilitates the capacitive radio frequency rotary heating mechanism to closely adhere to heat.

In one embodiment, on the global irradiation ring 33, one or more pairs of capacitor plates 36 and a number of microwave magnetrons 37 are arranged in different planes, that is, the ring surface on which one or more pairs of capacitor plates 36 are located and the ring surface on which the several microwave magnetrons 37 are located are partially or completely staggered on the central axis of the global irradiation ring 33 to avoid coplanarity.

### Embodiment 4

FIG. 17 is a schematic diagram of two global irradiation rings 33 provided with a precise high heat global scalding cancer killing device in the body. As shown in FIG. 17, unlike the microwave irradiation mechanism and the capacitive radio frequency irradiation mechanism in the embodiment shown in FIG. 15 that are jointly arranged on the same global irradiation ring 33, in this embodiment, the microwave rotary heating mechanism is arranged on the first irradiating ring 33a and the capacitive radio frequency rotary heating mechanism is arranged on the second irradiating ring 33b, and the two rotate independently relative to each other.

### Embodiment 5

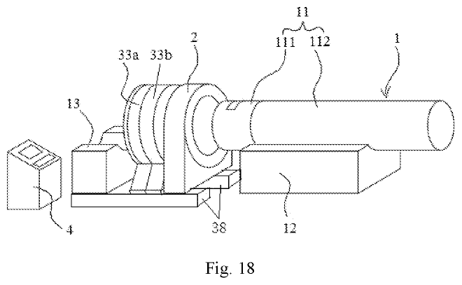

FIG. 18 is a modified structure of the accurate high heat global scalding cancer killing device in the body. As shown in FIG. 18, unlike the fixed layout of the temperature measuring mechanism 2 and the global irradiation unit 3 in the embodiment shown in FIG. 15, in this embodiment, the temperature measuring mechanism 2 and the global irradiation unit 3 are jointly arranged on the guide rail 38, It can slide back and forth along the guide rail, which is convenient for the mobile temperature measurement.

Described above are only preferred embodiments of the present disclosure, which are not intended to limit the scope of the present disclosure. It should be understood that any variations, modifications and replacements made by those skilled in the art without departing from the spirit of the disclosure shall fall within the scope of the disclosure defined by the appended claims.

What is claimed is:

1. A global irradiation thermotherapy system based on coordinated rotation of microwave and radio frequency, comprising:
an annular phantom;
an intelligent control unit;
a microwave rotary heating mechanism;
a capacitive radio frequency rotary heating mechanism; and
a temperature measuring mechanism;
wherein the microwave rotary heating mechanism, the capacitive radio frequency rotary heating mechanism and the temperature measuring mechanism are connected to the intelligent control unit; the capacitive radio frequency rotary heating mechanism is configured to rotationally heat each point in an area of a human body with a lateral radius of 0˜13 cm through the annular phantom to form a circular high-temperature zone, wherein a temperature of the circular high-temperature zone decreases from center to outside, and fat tissues in an outermost layer of the human body has the lowest temperature;
the annular phantom is adapted to the capacitive radio frequency rotary heating mechanism, and comprises a liquid-injection annular capsule, a cylindrical rib frame located at an inner side of an outer surface of the liquid-injection annular capsule, and a hoop located outside two ends of the liquid-injection annular capsule along an axial direction; an outer ring surface of the liquid-injection annular capsule is in rotational contact with a radio frequency electrode plate of the capacitive radio frequency rotary heating mechanism; and the outer ring surface of the liquid-injection annular capsule is made of a flexible insulating material, and is supported by the cylindrical rib frame and the hoop to maintain a cylindrical shape; an inner ring surface of the liquid-injection annular capsule has a soft and elastic structure; when the liquid-injection annular capsule is empty, the inner ring surface of the liquid-injection annular capsule constricts to fit an inner side of the outer ring surface of the liquid-injection annular capsule; and when the liquid-injection annular capsule is filled with a liquid, the inner ring surface of the liquid-injection annular capsule fits and surrounds skins of the human body to play a role as a transition medium between the radio frequency electrode plate and the skins of the human body;
the microwave rotary heating mechanism is configured to form an annular high-temperature layer with a thickness of 2˜4 cm on an inner side of a fat tissue through rotary microwave irradiation, and a temperature of the high-temperature layer decreases from outside to inside;
the temperature measuring mechanism is configured to obtain precise temperature data of several key points in an accessible part of a thermotherapy area of the human body via optical-fiber temperature-measuring technology as a reference system, and to accurately measure a temperature of each point in the thermotherapy area based on combination of nuclear magnetic thermal imaging technology; and
the intelligent control unit is configured to coordinately control the microwave rotary heating mechanism and the capacitive radio frequency rotary heating mechanism to rotationally irradiate the thermotherapy area to perform deep-shallow layered and complementary diathermy, and to perform precise temperature control based on real-time temperature measurement of the temperature measuring mechanism to achieve uniform and precise hyperthermia throughout the thermotherapy area.

2. The global irradiation thermotherapy system of claim 1, wherein a microwave frequency of the microwave rotary heating mechanism is 300 MHz˜3000 MHz, and a radio frequency of the capacitive radio frequency rotary heating mechanism is 1 MHz˜300 MHz.

3. The global irradiation thermotherapy system of claim 1, wherein the temperature measuring mechanism comprises a nuclear magnetic thermal imaging device, an optical fiber temperature measuring device and a data processing platform;
the nuclear magnetic thermal imaging device is configured to establish a temperature field distribution map library of hot points respectively corresponding to individual temperatures according to nuclear magnetic resonance scanning data, and to obtain a temperature field distribution map of the thermotherapy area of the human body by scanning;
the optical fiber temperature measuring device is configured to obtain temperatures of several key points in the accessible part of the thermotherapy area as temperature calibration points by using a non-invasive technology;
the data processing platform is configured to calculate the temperature field distribution map scanned by the nuclear magnetic thermal imaging device based on the temperature field distribution map library to obtain a temperature scanning data of each hot point in the thermotherapy area, and is configured to calibrate the temperature scanning data of each hot point using temperature data of the temperature calibration points as reference to obtain accurate temperature data of each hot point in the thermotherapy area.

4. The global irradiation thermotherapy system of claim 3, wherein the optical fiber temperature measuring device comprises a temperature data acquisition unit, a temperature data processing unit and a sensing optical fiber; and a plurality of optical fiber sensors are provided on the sensing optical fiber.

5. The global irradiation thermotherapy system of claim 1, wherein the cylindrical rib frame consists of two C-shaped structures to facilitate opening and closing of the liquid-injection annular capsule.

6. The global irradiation thermotherapy system of claim 1, wherein the hoop consists of two C-shaped structures; the hoop is fixedly sleeved on outer sides of two ends of the liquid-injection annular capsule, respectively; and the hoop is configured to be opened and closed together with the liquid-injection annular capsule.

7. The global irradiation thermotherapy system of claim 1, wherein the inner ring surface of the liquid-injection annular capsule has a spliced structure, and is composed of a first flexible layer and a middle elastic layer and a second flexible layer along an axial direction of the liquid-injection annular capsule; and the middle elastic layer is configured to fit and surround the skins of the human body after being filled with the liquid.

8. The global irradiation thermotherapy system of claim 1, wherein
the outer ring surface of the liquid-injection annular capsule is cylindrical, and is configured to be compressed by the radio frequency electrode plate to increase a contact area.

9. The global irradiation thermotherapy system of claim 1, wherein the liquid-injection annular capsule is composed of two C-shaped closed capsules, which are arranged in an up-and-down manner to surround a body cavity; both ends of the liquid-injection annular capsule along the axial direction are respectively provided with a ring-shaped soft cloth with a contractile inner opening; and the liquid filled in the liquid-injection annular capsule is a non-polar liquid.

10. The global irradiation thermotherapy system of claim 1,
further comprising:
a translational heat-preservation bin;
wherein the translational heat-preservation bin is configured to carry the human body in and out of an internal space of the microwave rotary heating mechanism and the capacitive radio frequency rotary heating mechanism.

11. The global irradiation thermotherapy system of claim 1, further comprising:
a rotating mechanism;
wherein the rotating mechanism is configured to drive a global irradiation ring equipped with the capacitive radio frequency rotary heating mechanism or/and the microwave rotary heating mechanism to rotate or brake according to a control program.

12. A method for measuring a temperature of a thermotherapy area of a human body using the global irradiation thermotherapy system of claim 3, comprising:
(S401) scanning hot points using the nuclear magnetic thermal imaging device to obtain thermal imaging data, and establishing a temperature field distribution map library of the hot points respectively corresponding to individual temperatures;
(S402) obtaining the temperature field distribution map of the thermotherapy area of the human body by scanning using the nuclear magnetic thermal imaging device;
(S403) obtaining, by the optical fiber temperature measuring device, temperatures of several key points in the accessible part of the thermotherapy area as temperature calibration points by using a non-invasive technology; and
(S404) calculating the temperature field distribution map obtained in step (S402) based on the temperature field distribution map library obtained in step (S401) to obtain a temperature scanning data of each point in the thermotherapy area; and calibrating the temperature scanning data of each point in the thermotherapy area using temperature data of the temperature calibration points as a reference to obtain accurate temperature data of each point in the thermotherapy area.

13. The method of claim 12, wherein in step (S401), the temperature field distribution map library comprises thermal imaging data and temperature value of each hot point, and the thermal imaging data and temperature value of each hot point are in one-to-one correspondence.

14. The method of claim 12, wherein in step (S404), the temperature data of the temperature calibration points comprises accurate temperature value and location information; and the temperature field distribution map comprises thermal imaging information and location information of each point in the thermotherapy area; and the temperature calibration points have corresponding points in the temperature field distribution map.

15. A global thermotherapy instrument based on the global irradiation thermotherapy system of claim 1, comprising:
a global irradiation unit;
wherein the global irradiation unit comprises a microwave rotary heating mechanism, a capacitive radio frequency rotary heating mechanism, a rotating mechanism and an annular phantom; the microwave rotary heating mechanism and the capacitive radio frequency rotary heating mechanism are arranged on the same global irradiation ring or are respectively arranged on two global irradiation rings; the annular phantom is configured to make up for irregularity of the human body to form a regular cylinder, so as to allow the capacitive radio frequency rotary heating mechanism to fit the annular phantom to perform diathermy; the rotating mechanism is configured to drive the global irradiation ring to rotate; and based on combination of microwave rotary heating and capacitive radio frequency rotary heating, the global thermotherapy instrument achieves layered complementary diathermy, so as to achieve accurate and uniform hyperthermia in the entire thermotherapy area.

16. The global thermotherapy instrument of claim 15, wherein the temperature measuring mechanism is configured for real-time non-invasive temperature measurement of an object; and
the temperature measuring mechanism comprises an optical fiber temperature measuring device, a nuclear magnetic thermal imaging device and a data processing platform.

17. The global thermotherapy instrument of claim 15, further comprising:
a translational heat-preservation bin;
wherein the translational heat-preservation bin comprises a bin base, a bin body slidably arranged on the bin base, and a head holder; and the bin body passes through the nuclear magnetic thermal imaging device and the global irradiation unit in sequence to be supported on the head holder.

18. The global thermotherapy instrument of claim 17, wherein the nuclear magnetic thermal imaging device and the global irradiation unit are together arranged on a guide rail and are capable of sliding back and forth along the guide rail.

19. The global thermotherapy instrument of claim 15, wherein the microwave rotary heating mechanism is a microwave component with a microwave frequency of 300 MHz˜3000 MHz.

20. The global thermotherapy instrument of claim 15, wherein a radio frequency of the capacitive radio frequency rotary heating mechanism is 1 MHz˜300 MHz.
